(19) United States
(12) Patent Application Publication
(10) Pub. No.: US 2009/0306706 A1
(43) Pub. Date: Dec. 10, 2009

(54) OCCLUSION ELEMENT FOR OPENINGS IN THE HEART, IN PARTICULAR AN ASD OCCLUSION ELEMENT

(75) Inventor: Peter Osypka, Rheinfelden-Herten (DE)

Correspondence Address: VOLPE AND KOENIG, P.C., UNITED PLAZA, SUITE 1600, 30 SOUTH 17TH STREET, PHILADELPHIA, PA, 19103, US

(73) Assignee: Peter Osypka Stiftung Stiftung Des Burgerlichen Rechts, Grenzach-Wyhlen (DE)

(21) Appl. No.: 12/373,532

(22) PCT Filed: Jul. 7, 2007
(86) PCT No.: PCT/EP2007/006033

§ 371 Date: Jan. 13, 2009

(30) Foreign Application Priority Data
Aug. 29, 2006 (DE) 10 2006 040 415.7

(51) Int. Cl. A61B 17/03

(52) U.S. Cl. 606/213

(57) ABSTRACT

An occlusion element (1) for unwanted openings in the heart is provided. The braid forming the occlusion element (1) is radially constricted in a middle area (3) that passes through the opening in the heart when it is in the position of use, such that, on both sides of this constricted middle area (3), braided occlusion discs (4) and (5) are formed, which engage on both sides over the edges of an unwanted opening in the heart in the position of use. In the starting position, prior to the constriction of the middle area (3), a tubular braid is formed with a double wall along at least part of its axial extent, having a first braid portion (6) of smaller cross section surrounded at a distance on the outside by a second braid portion (7) of greater cross section. The filaments or wires (2) of the two braid portions (6) and (7) continue into one another at a common end face (8) or are connected in one piece, such that the constriction or compression results in at least one smooth, double-walled occlusion disc (7) whose outside wall is formed by the common end face (8) or includes the latter.

### BACKGROUND

The invention relates to an occlusion element for undesired openings in the heart, in particular, to an ASD occlusion element or occlusion instrument that is braided from shape-memory material filaments, in particular, from shape-memory synthetic filaments, from nitinol strands, or from strands or filaments that are sheathed with plastic and that are made from shape-memory material, wherein this fabric forming the occlusion element is constricted or compressed in the radial direction in the middle region passing through the opening in the heart in the position of use, so that on both sides of this constricted or compressed region there are braided occlusion disks or regions that overlap the edges of the undesired opening in the heart on both sides in the position of use.

Such an occlusion element braided from nitinol strands is known from WO 99/12478 A1. An originally approximately cylindrical fabric is flattened centrally in the radial direction and the two ends of the fabric are each joined by a sleeve or connection piece gripping the individual strands with a smaller diameter and fixed in the corresponding shape, so that, for example, for an occlusion element arranged between the two cardiac atriums, such a sleeve also projects into the left atrium, where arterial blood generates a relatively high pressure and can lead to disruptions like those described in DE 10 338 702 B3, in particular in paragraph

From this publication, embodiments are also known in which the sleeves or connection pieces are inserted into the interior of the fabric. Therefore, they indeed no longer project outward, but the entire occlusion element also has only a relatively low stability especially in the axial direction due to these holding sleeves arranged in the interior close to the front ends.

From DE 10 338 702 B3 an occlusion element of the type defined above is known in which there is only one such sleeve or holder for the ends of the strands of the fabric and a second such sleeve is eliminated, so that this occlusion element can be inserted such that there is no such sleeve in the left atrium of the heart. However, in this way the braided occlusion disk to be brought into the left atrium or arranged in the position of use has only one layer, that is, the somewhat curved or disk-like fabric reaches from the inner constriction or the region of smaller cross section outward and ends there. This requires special precautions, so that this occlusion disk with only one layer cannot be possibly pushed through the undesired opening due to the higher blood pressure prevailing in the left atrium. For example, a correspondingly narrow and accordingly more expensive fabric must be provided and/or this one-layer occlusion disk must be over-dimensioned and have a larger construction than actually required by the undesired opening in the heart or there must be some reinforcement on this occlusion disk.

From WO 01/21246 A1, an occlusion element formed from a fabric is known in which one front end is inserted and a fabric part is guided with its end through the other front end, wherein optionally rings are arranged at the ends and the one fabric part can be held in the distal end of the other fabric part. This also does not produce high stability in the axial direction, because the opposite position of the penetrating fabric parts is not unique and is not fixed before use.

### SUMMARY

Therefore there is the task to create an occlusion element of the type defined above in which the advantage is maintained so that there is no sleeve or holder for the filaments or strands of the fabric at least in the left atrium of a heart, but nevertheless the occlusion disk to be arranged in the left atrium has sufficient stiffness or stability, in order to be able to withstand the higher blood pressure prevailing there, without having to be, for example, overdimensioned. In addition, the occlusion element should have good stability in the axial direction.

For achieving this apparently contradictory task, the occlusion element defined above is characterized in that, in the starting position before the constriction of the middle region, the somewhat tubular fabric has a double-walled construction or configuration across its entire axial extent, that is, it has a first fabric section with smaller cross section and a second fabric section with greater cross section surrounding this first fabric section on the outside, wherein the two fabric sections extend across the entire axial extent of the occlusion element and transition one into the other at a common front end or the filaments or strands forming them are continuous or are connected, so that through the constriction or compression, a projection-free, double-walled occlusion disk is formed whose outer wall comprises the common front end or is formed by this common front end, the somewhat tubular first fabric section of smaller and especially circular cross section at its free end—usually at the end where the mesh begins—has a sleeve or a holder and the filaments or strands of the fabric are fixed by this sleeve or holder, and the second fabric section of larger and especially circular cross section extending opposite this first fabric section on the outside is guided together with its filaments or strands on the front end also to this holder or sleeve for fixing these filaments or strands in a somewhat radial direction.

Thus, a fabric is used whose profile is continuous from an inner section of smaller diameter outward by a front end-side change in direction and is guided further in the opposite direction, so that a single, but double-walled fabric with two concentric fabric sections essentially forms the entire occlusion element and no joining sleeve is required for the filaments or strands accordingly at one of its front ends. Furthermore, in this way a doubling of the layers of the fabric is produced in the region of its radial extension from the first inner fabric section to the outer fabric section and from there back to the constriction, so that the occlusion disk formed in this way has at least two layers.

Therefore, because the fabric is initially guided in one axial direction and then back outward at one front end under expansion of the cross section of this fabric, in this front end region there is at least one double-walled and double sleeve-like structure with an initially central opening through the first inner fabric section. This opening can be closed by the common sleeve or holder at the beginning of this first fabric section in a way still to be explained, wherein this sleeve is generated or can be formed, for example, somewhat similarly to that known from DE 10 338 702 B3.

Because through a braiding procedure that is continuous in opposing directions, the holder of the individual strands located on the braiding machine or device is always led back into its first starting position or even, in addition, the entire production procedure of such occlusion elements and here the braiding procedure can be simplified and made economical and it is even conceivable, after production of a double-walled fabric, to guide the filaments or strands coming in at its end initially unbraided into an original starting position and from there, in turn, to produce the next double-walled fabric, so that a series production of such occlusion elements is enabled by the double-walled arrangement according to the invention of fabric sections connected to each other, without having to realign the braiding device after the production of each occlusion element, as is required in the solution according to DE 10 338 702 B3. Thus, despite the apparently greater expense of a double-walled fabric and despite the apparently greater material consumption favorable for the occlusion element, the occlusion element according to the invention has the additional advantage of allowing a favorable and economical series production. Simultaneously, the safety of the occlusion element is also increased in the left atrium of a heart.

The occlusion element is especially advantageous as an ASD occlusion, but can also be used as a PDA or VSD occlusion, wherein the advantage is also used so that no holding sleeve for the filaments or strands projects at one front end of the occlusion element and both occlusion disks can have a double-layer construction and have an approximately matching strength and large stability also in the axial direction.

Here, one can manage with only one sleeve or holder for the filaments or strands of the entire fabric, wherein then, however, the first fabric section has a correspondingly larger length reaching across the entire axial extent of the occlusion element already in the original position before the constriction and flattening of this occlusion element, and is flattened at the same time.

Here it is useful when at least the second fabric section that continues from the first inner fabric section and overlaps on the outside especially coaxially and concentrically and at a distance to the first inner fabric section across the common front end, and an occlusion disk constriction in a middle axial region and the entire occlusion element can be flattened in the axial direction. This produces, on both sides of the constricted position, the desired occlusion disks, wherein their axial dimensions can be kept small by the flattening, but simultaneously a sufficiently large radial extent is also achieved for gripping over the edges of the opening in the heart.

The diameter of the second fabric section can be greater than that of the first fabric section in front of the constriction by approximately five times to fifteen times, in particular, approximately ten times. In this way it is achieved that primarily the occlusion disks are formed predominantly by the larger second fabric section that, however, then both have at least a two-layer construction.

Here it is favorable when in the position of use the inner and the outer fabric section are flattened on both sides of the constriction in the axial direction and therefore enlarged in the radial direction and the two occlusion disks formed in this way have, in total, a four-layer construction in its middle part or center region seen in the axial direction and a two-layer construction in the radial, outer region outside of the inner fabric section. If a fabric in which the first and the second fabric section initially reaches across the entire axial extent is flattened, both fabric sections are flattened, wherein the inner fabric section leads to correspondingly smaller disk-shaped structures due to its smaller radial extent, while the second fabric section also leads to considerably larger occlusion disks that form the essential parts of the occlusion element due to its considerably larger extent in the flattened state.

The sleeve or holder joining the filaments or strands can have internal threading for a delivery catheter or for a manipulation tool on their front end or end face pointing outward axially. Therefore, the single sleeve can be used advantageously for feeding or manipulating the device.

Primarily in the combination of individual or several of the previously described features and measures, an occlusion element is produced in which occlusion disks arranged on both sides of an undesired opening have at least a two-layer construction, so that one can also work with thinner filaments or strands that are accordingly less stiff and can therefore simplify the required deformations in a way accommodating the anatomy, for example, in the heart and also for the production, as well as later for the introduction of the occlusion element with the help of a delivery catheter, wherein only a single sleeve or holder is required for fixing the filaments or strands.

### BRIEF DESCRIPTION OF THE DRAWINGS

Below, an embodiment of the invention is described in greater detail with reference to the drawing. Shown in a partially schematic diagram are:

FIG. 1 is a view of an occlusion element formed of a first inner fabric section of smaller diameter that transitions at one front end by the strands or filaments forming it transitioning into an outer second fabric section of larger diameter, wherein both fabric sections are arranged coaxially and concentric to each other and has at the front end opposite the common front end, a common sleeve or holder for the strands or filaments forming them,

FIG. 2 is a schematic representation of the constriction of initially the second outer fabric section,

FIG. 3 is a view of the occlusion element after the constriction and flattening in the axial direction, wherein the filaments and fabric parts still project past the common sleeve or holder in the axial direction, and also

FIG. 4 is a view of the deployed occlusion element with a common sleeve projecting on one front end of the occlusion element for holding together the filaments or strands, while on the front end facing away, no such sleeve projects and both occlusion disks of this occlusion element also have at least a double-walled construction in their radial outer regions.

### DETAILED DESCRIPTION OF THE PREFERRED EMBODIMENTS

An occlusion element designated overall with 1 and shown primarily in FIG. 4 is provided for insertion into undesired openings in the heart as an ASD occlusion element or occlusion instrument, but could also be used as a PDA or VSD occlusion.

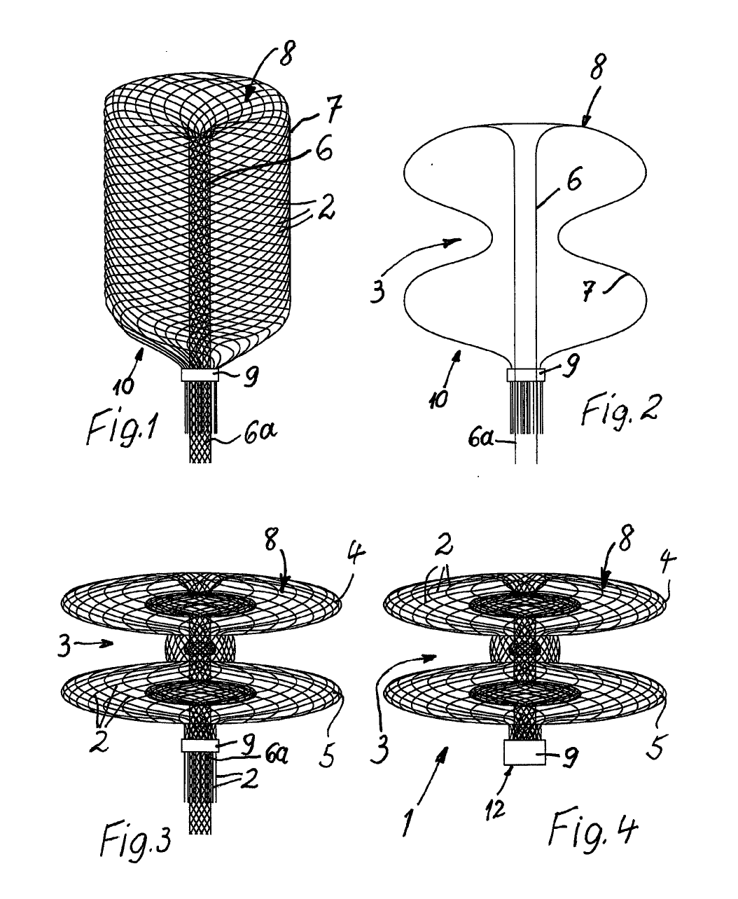

This occlusion element 1 is braided from shape-memory material filaments 2, for example, made from nitinol filaments or from strands or filaments sheathed with plastic or also from shape-memory plastic filaments, which can be seen particularly well in FIG. 1, but also in FIGS. 3 and 4.

Here, this fabric that forms the occlusion element 1 and that can be seen particularly well in FIG. 1 is radially constricted or compressed in a middle region 3 passing through the opening in the heart in the position of use according to FIGS. 2 to 4, so that braided occlusion disks 4 and 5 or regions are formed on both sides of this constricted or compressed middle region 3, wherein these disks or regions overlap the edges of the undesired opening in the heart on both sides in the position of use, as is known, for example, from FIG. 16 of U.S. Pat. No. 5,725,552 and furthermore from DE 10 338 702 B3.

Here it is provided according to FIG. 1 that the somewhat tubular fabric in the starting position before the constriction of the middle region 3 has a double-walled construction across the entire axial extent, that is, it has a first fabric section 6 with smaller cross section and a second fabric section 7 with larger cross section surrounding or overlapping the first section on the outside.

Here, one sees primarily in FIG. 1, but also clear in the other figures, that the two fabric sections 6 and 7 transition one into the other at a common front end 8 or continue or are connected, that is, the filaments or strands 2 transition integrally from the first fabric section 6 into the second fabric section 7 of larger diameter, wherein this radial transition forms the front end 8 of the entire fabric or occlusion element 1.

In this way, a projection-free, at least double-walled occlusion disk 4 whose outer wall comprises the common front end 8 or corresponds to this front end 8, as is indicated primarily in FIGS. 2 to 4, is formed by the constriction or compression of the middle region 3.

Simultaneously, one sees in the figures that there is no projection extending or projecting outward in the axial direction with a holder or sleeve for the filaments or strands 2 on this occlusion disk 4.

According to FIG. 3, at least the second fabric section 7 that continues from the first inner fabric section and that overlaps on the outside coaxially and at a distance to the first inner fabric section 6 across the common front end 8, forms an occlusion disk 4 and is constricted in the axial middle region 3, and according to FIGS. 3 and 4, the entire occlusion element 1 is flattened in the axial direction, so that the dimensions originally provided according to FIG. 1 for the axial extent are considerably smaller for the completed occlusion element 1, while simultaneously its radial dimensions or extent is increased relative to the original blank. Here, the two occlusion disks 4 and 5 have approximately matching dimensions.

Here, one sees that the diameter of the second fabric section 7 before the constriction is approximately five times to fifteen times, and in the preferred embodiment, approximately ten times as large as that of the first fabric section 6, that is, in the position of use, primarily the second fabric section 7 forms the occlusion disks 4 and 5, wherein, however, the first fabric section 6 has the important task of holding together and gripping the filaments or strands 2 of the occlusion element 1, without requiring a projection with a holding sleeve on the front end 8 and thus the occlusion disk 4 in the axial direction.

This is achieved in that the somewhat tubular first fabric section 6 of smaller and especially circular cross section has a sleeve 9 or holder, in the following also referred to only as “sleeve 9,” at its free end 6a from where the braiding procedure starts for forming the fabric, and the filaments or strands 2 of the fabric are fixed by the sleeve 9, wherein the dimensions of the cross section or diameter of this sleeve 9 do not or only slightly exceed those of the fabric section 6, and the sleeve 9 can also be constructed as a completely filled connection piece analogous to the construction according to DE 10 338 702 B3.

The second fabric section 7 of larger and especially circular cross section running on the outside opposite this first fabric section 6 is guided together with its filaments or strands 2 on the front end, that is, on the front end 10 opposite the front end 8, also into the holder or sleeve for fixing these filaments or strands 2 in an approximately radial direction, so that all of the filaments or strands 2 of the fabric are fixed, so that the braiding cannot loosen, wherein the sleeve could form with the filaments or strands a filled connection piece.

According to FIG. 1 it is here provided that the sleeve 9 for the filaments or strands 2 of the first fabric section 6 also contains, holds together, or has the filaments or strands 2 of the second fabric section 7 and that both fabric sections 6 and 7 extend across the entire axial extent of the occlusion element 1. One could also say that the sleeves are connected for joining the strands 2 of the first fabric section 6 and the strands 2 of the second fabric section 7 integrally into a single sleeve 9.

Through this arrangement, an occlusion element 1 according to FIGS. 3 and 4 is produced in which, in the position of use, the inner and the outer fabric sections 6 and 7 are flattened on both sides of the constriction in the middle region 3 in the axial direction and are therefore increased in the radial direction and the two occlusion disks 4 and 5 formed in this way have, overall, a four-layer construction in their center region viewed in the axial direction and a two-layer construction in the radially outer region outside of the inner fabric section 6. Due to the flattening according to FIGS. 3 and 4, namely, also the inner fabric section 6 is flattened accordingly, which, however, is essentially prevented at the constriction in the middle region 3, so that the axial shortening both of the inner first fabric section 6 and also that of the outer second fabric section 7 leads to corresponding radial expansions and thus to the construction of the mentioned occlusion disks 4 and 5 at these fabric sections 6 and 7.

In a way not shown in greater detail, the common sleeve 9 can have an internal filament for a delivery catheter or for a manipulation tool used for moving and positioning the occlusion element 1 on its front end or end face 12 pointing axially outwardly toward the front end 10.

The occlusion element 1 for undesired openings in the heart that can be used primarily as an ASD occlusion element or occlusion instrument is braided from shape-memory material filaments 2, for example, from plastic filaments, nitinol strands, or from strands or filaments that are made from shape-memory material and that are sheathed with plastic. This fabric forming the occlusion element 1 is constricted or compressed radially or dimensioned smaller in a middle region 3 passing through the opening in the heart in the position of use, so that braided occlusion disks 4 and 5 or occlusion regions that overlap the edges of an undesired opening in the heart in the position of use on both sides are provided on both sides of this constricted, compressed, or smaller dimensioned middle region 3. Here, in the starting position before the constriction of the middle region 3, a tubular fabric that has a double-walled construction across at least one part of its axial extent, that is, a first fabric section 6 with smaller cross section and a second fabric section 7 with larger cross section surrounding this first fabric section on the outside at a distance is provided, wherein the filaments or strands 2 of the two fabric sections 6 and 7 are continuous or are connected integrally at a common front end 8, so that the one or more projection-free, double-walled occlusion disk 4 whose outer wall is formed by the common front end 8 or comprises this front end is formed by the constriction or compression.

1. Occlusion element (1) for undesired openings in a heart, comprising a braided fabric of shape-memory material filaments or strands (2), the fabric forming the occlusion element (1) is radially constricted or compressed in an axial middle region (3) passing through an opening in the heart in a position of use, so that braided occlusion disks (4, 5) or regions that overlap edges of the undesired opening in the heart on both sides in the position of use are provided on both sides of the middle region (3), the fabric is generally tubular and has a double-walled construction across an entire axial extent thereof in an original position before constriction or compression of the middle region (3), such that a first fabric section (6) with a smaller cross section and a second fabric section (7) with a larger cross section surrounds the first section on an outside thereof, the two fabric sections (6, 7) extend across an entire axial extent of the occlusion element (1) and transition one into the other at a common front end (8) or are continuous or are connected to each other, so that through the constriction or compression a projection-free, double-walled occlusion disk (4) is formed whose outer wall comprises the common front end (8), the first fabric section (6) having the smaller cross section has at its free end (6a) a sleeve (9) or a holder and the filaments or strands (2) of the fabric are fixed by the sleeve (9) or holder, and the second fabric section (7) having the larger cross section extending on the outside opposite the first fabric section (6) is also guided together with its filaments or strands (2) in an approximately radial direction at a front end likewise toward the holder or sleeve (9) for fixing the filaments or strands (2).

2. The occlusion element according to claim 1, wherein at least the second fabric section (7) that continues from the first inner fabric section and overlaps on the outside, coaxially and at a distance to the first inner fabric section (6) across the common front end (8) and the occlusion disk (4) is constricted in the axial middle region (3) and the entire occlusion element (1) is flattened in the axial direction.

3. The occlusion element according to claim 1, wherein before the constriction, a diameter of the second fabric section (7) is approximately five times to fifteen times greater than the diameter of the first fabric section (6).

4. The occlusion element according to claim 1, wherein the first fabric section (6) and the second fabric section (7) each have a circular cross section.

5. The occlusion element according to claim 1, wherein in the position of use, the inner and the outer fabric sections (6, 7) are flattened in the axial direction on both sides of the constriction (3) and are therefore enlarged in a radial direction, and the two occlusion disks (4, 5) formed have, overall, a four-layer construction at least in a middle part or center region viewed in the axial direction and a two-layer construction in a radially outer region outside of the inner fabric section (6).

6. The occlusion element according to claim 1, wherein the sleeve (9) or holder joining the filaments or strands (2) has an internal filament or a contact point for a delivery catheter or a manipulation tool on a front end or end face (12) pointing outwardly in an axial direction.

7. The occlusion element according to claim 1, wherein the shape-memory material filaments or strands (2) comprise at least one of shape-memory plastic filaments, nitinol strands or strands or filaments made from shape-memory material sheathed with plastic.

8. The occlusion element of claim 1, wherein the occlusion element is adapted for closing an atrial septal defect.
